(19) United States
(12) Patent Application Publication
(10) Pub. No.: US 2005/0113659 A1
(43) Pub. Date: May 26, 2005

(54) Device for data input for surgical navigation system

(76) Inventors: Albert Pothier, Memphis, TN (US); Daniel McCombs, Memphis, TN (US)

Correspondence Address: CHIEF PATENT COUNSEL;SMITH & NEPHEW, INC., 1450 BROOKS ROAD, MEMPHIS, TN, 38116, US

(21) Appl. No.: 10/997,052

(22) Filed: Nov. 24, 2004

Related U.S. Application Data
(60) Provisional application No. 60/525,346, filed on Nov. 26, 2003.

(51) Int. Cl. A61B 17/00

(52) U.S. Cl. 600/372; 606/1; 600/414

(57) ABSTRACT

A probe for data input for a computer-assisted surgical system, comprising a body; one or more movable fiducials operably associated with the probe; and one or more stationary fiducials operably associated with the probe; wherein the movement of the one or more movable fiducials relative to the one or more of the stationary fiducials triggers data input on a position or an orientation of the probe, or both. The probe is particularly suitable for minimally invasive surgery. Also provided is a method of using the probe to input data during computer-assisted surgery.

### CROSS-REFERENCE TO RELATED APPLICATION

This application claims priority to U.S. Provisional Patent Application Ser. No. 60/525,346 entitled “Device for Data Input for Surgical Navigation System” filed on Nov. 26, 2003, the entire content of which is incorporated herein.

### FIELD OF INVENTION

The invention relates generally to computer-assisted surgical (CAS) systems and methods of their use. More specifically, the invention relates to instrumentation, systems, and processes for tracking anatomy, implements, instrumentation, trial implants, implant components and virtual constructs or references, and inputting data related to them in connection with surgical procedures, including, but not limited to, orthopedic surgical procedures, such as joint replacement surgeries. Probes associated with referencing devices or fiducials, and devices for inputting probe location and/or orientation data.

### BACKGROUND AND SUMMARY

Computer-assisted surgical systems use various imaging and tracking devices and combine the image information with computer algorithms to track the position of the patient's anatomy, surgical instruments, prosthetic components, virtual surgical constructs such as body and limb axes, and other surgical structures and components. The computer-assisted surgical systems use this data to make highly individualized recommendations on a number of parameters, including, but not limited to, patient's positioning, the most optimal surgical cuts, and prosthetic component selection and positioning. Orthopedic surgery, including, but not limited to, joint replacement surgery, is one of the areas where computer-assisted surgery is becoming increasingly popular.

During joint replacement surgery, diseased or damaged joints within the musculoskeletal system of a human or an animal, such as, but not limited to, a knee, a hip, a shoulder, an ankle, or an elbow joint, are partially or totally replaced with long-term surgically implantable devices, also referred to as joint implants, joint prostheses, joint prosthetic implants, joint replacements, or prosthetic joints.

Some of the computer-assisted surgery systems use imaging systems based on CT scans and/or MRI data or on digitized points on the anatomy. Other systems align preoperative CT scans, MRIs, or other images with intraoperative patient positions. A preoperative planning system allows the surgeon to select reference points and to determine the final implant position. Intraoperatively, the computer-assisted surgery system calibrates the patient position to that preoperative plan, such as by using a “point cloud” technique, and can use a robot to make bone preparations. Other systems use position and/or orientation tracking sensors, such as infrared sensors acting stereoscopically or otherwise, to track positions of body parts, surgery-related items such as implements, instrumentation, trial prosthetics, prosthetic components, and virtual constructs or references such as rotational axes which have been calculated and stored based on designation of bone landmarks.

Processing capability such as any desired form of computer functionality, whether standalone, networked, or otherwise, takes into account the position and orientation information as to various items in the position sensing field (which may correspond generally or specifically to all or portions or more than all of the surgical field) based on sensed position and orientation of their associated fiducials or based on stored position and/or orientation information. The processing functionality correlates this position and orientation information for each object with stored information regarding the items, such as a computerized fluoroscopic imaged file of a bone, a wire frame data file for rendering a representation of an instrumentation component, trial joint prosthesis or actual joint prosthesis, or a computer generated file relating to a rotational axis or other virtual construct or reference. The processing functionality then displays position and orientation of these objects on a screen or monitor, or otherwise. The surgeon may navigate tools, instrumentation, prosthetic components, actual prostheses, and other items relative to bones and other body parts to perform a surgery more accurately, efficiently, and with better alignment.

The computer-assisted surgical systems use the position and/or orientation tracking sensors to track the fiducial or reference devices associated with the body parts, surgery-related items such as implements, instrumentation, trial prosthetics, prosthetic components, and virtual constructs or references, such as limb rotational axes calculated and stored based on designation of bone landmarks. Any or all of these may be physically or virtually associated with any desired form of mark, structure, component, or other fiducial or reference device or technique that allows position or orientation, or both, of the associated item to be sensed and tracked in space, time, or both. Fiducials can be single markers or reference frames containing one or more reference elements. Reference elements can be active, such as energy emitting, or passive, such as energy reflective or absorbing, or any combination thereof. Reference elements may be optical or employ any suitable form of electromagnetic energy, such as infrared, micro or radio waves. In general, any other suitable form of signaling may also be used, as well as combinations of various signals. To report position and orientation of the item, the active fiducials, such as microchips with appropriate field or a position/orientation sensing functionality, and a communications link, such as a spread-spectrum radio frequency link, may be used. Hybrid active/passive fiducials are also possible. The output of the reference elements may be processed separately or in concert by the processing functionality.

To locate and register an anatomical landmark, a CAS system user may employ a probe operatively associated with one or more fiducials. The one or more fiducials provide information relating the landmark via a tracking/sensing functionality to the processing functionality. To indicate input of a desired point to the processing functionality, one or more devices for data input are commonly incorporated into the computer-assisted surgery systems. The data input devices allow the user to communicate to the processing functionality to register data from the probe-associated fiducials.

The processing functionality generally comprises a computer functionality. A CAS system user may input data to a computer functionality by a variety of means. Some systems employ a conventional computer interface, such as a keyboard or a computer mouse, or a computer screen with a tactile interface. In some systems, the user presses a foot pedal to indicate to the computer to input probe location data. Others use a wired keypad or a wireless handheld remote.

For many applications it is desirable to eliminate the use of wires or electricity to communicate a data input signal to the computer. Wireless and powerless probes are generally easier to manufacture and manipulate and are more reliable. In other applications, it is desirable to eliminate or decrease radio or other strong electromagnetic signals to avoid interferences within the CAS system. One example of a wireless, powerless data input device is a probe incorporating reflective spheres that are tracked by the infrared cameras. To communicate to the computer functionality to enter a point on the anatomy, the probe tip is held at the desired point, and the body of the probe is moved in an arc motion. The motion is read by the computer and the point is accepted. Although the probe is conveniently wireless and independent of electricity, the required ‘arcing’ motion is often difficult for the user to accomplish, particularly in the context of minimally invasive surgery.

The term “minimally invasive surgery” (MIS) generally refers to the surgical techniques that minimize the size of the surgical incision and trauma to tissues. Minimally invasive surgery is generally less intrusive than conventional surgery, thereby shortening both surgical time and recovery time. Minimally invasive techniques are advantageous over conventional techniques by providing, for example, a smaller incision, less soft-tissue exposure, and minimal trauma to the tissues. To achieve the above goals of MIS, it is necessary to modify the traditional surgical techniques and instruments that require long surgical cuts and extensive exposure of the patient's tissues. The motion of a probe required to communicate data input signal to the computer may require an incision larger than a minimally invasive surgical incision and may lead to unnecessary trauma to the patient's tissues.

In view of the foregoing, probes, reference, or data input devices are desirable that eliminate the use of wires or electricity to communicate a data input signal to a computer functionality during computer-assisted surgery. Also desirable are the probes that are easier to manufacture and manipulate, and are more reliable. Also needed are the probes, reference, or data input devices adapted for minimally invasive applications is also needed. More specifically, such adaptations are directed towards minimizing surgical incisions required for operating the probe and trauma to patient's tissue, thereby improving the patient's recovery.

### SUMMARY

The aspects and embodiments of the present invention provide improved devices for data input during computer-assisted surgery. Computer-assisted surgery systems comprising improved data input devices are also provided, as well as methods of use of the improved devices, particularly in the contexts of computer assisted surgery and minimally invasive surgery.

In a preferred embodiment, the device for data input is a probe, comprising a body, one or more markers or fiducials operatively associated with the probe and movable relative to the body of the probe, and one or more markers or fiducials operatively associated with the probe and stationary relative to the body of the probe. The movement of the one or more movable markers or fiducials relative to the one or more stationary markers or fiducials triggers data input to the CAS system.

A computer-assisted surgical system, also referred to as a surgical navigation system, comprises a tracking functionality or sensor for tracking the position and/or orientation of the one or more of markers or fiducials. The tracking or sensing functionality senses the signal from the probe and communicates it to the processing functionality. The processing functionality registers the location/orientation of the probe, continuously or when prompted by the user. A movement of the one or more markers of the fiducials relative to the one or more stationary markers or fiducials signals to the processing functionality that the user of the probe is seeking to give input on the location of the probe to the computer functionality. The processing functionality interprets the movement of the one or more markers or fiducials as a signal to input data on the probe's location and/or orientation.

In one embodiment, the probe is advantageously adapted for minimally invasive surgery. In contrast to the conventional probes, to send the data input signal to the computer functionality, the user does not move the body of the probe within the surgical incision, avoiding unnecessary trauma to the patient's tissues. Also, to recognize a trigger signal from some of the conventional probes, at list the tip of the probe has to remain stationary while the fiducials move. This is required in order for the processing functionality to distinguish the trigger signal from regular movement from the probe, when both the tip and the fiducials move. In contrast, the improved probes do not require for the tip to remain stationary, thereby advantageously providing for types of data input, such as a surface or a cloud of points, where the tip moves to collect the data.

In a preferred embodiment, the probe is advantageously wireless and uses passive powerless fiducials, such as reflectors, thereby eliminating the use of wires, electricity, or radio waves. As compared to conventional devices, the probe is robust, easy and economical to manufacture, easy and intuitive to operate, and can withstand requisite sterilization procedures.

According to a preferred embodiment of the present invention, at least the following steps are involved in a method of data input using the improved probes:
1. Providing a probe comprising a body, one or more markers or fiducials operatively associated with the probe and movable relative to the body of the probe, and one or more markers or fiducials operatively associated with the probe and stationary relative to the body of the probe. A position and orientation of the fiducials can be tracked by the tracking functionality during computer-assisted surgery. The movement of the one or more movable markers or fiducials relative to the one or more stationary markers or fiducials triggers data input to the CAS system.
2. Locating the probe on a patient's body structure or other location desired to be input into the system.
3. Manipulating the probe to induce the movement of the one or more of the movable fiducials relative to the one or more stationary fiducials to indicate to the processing functionality that the user is seeking to input the probe's location and/or orientation.

The improved systems, devices and methods of their use may be used in any surgery, including but not limited to, orthopedic surgery, such as large bone orthopedic surgery and arthroscopy, total or partial joint repairs, reconstruction or replacement, including those of knees, hips, shoulders, elbows, ankles, and any other joint in the body, neurological surgery, such as, but not limited to, spine and cranial surgery, ear nose and throat (ENT) surgery, or any surgery where a part of a patient's anatomy can be generally stabilized relative to a fiducial. The probes according to aspects and embodiments of the present invention are used in conjunction with any or all of the imaging and position and/or orientation tracking sensors to present to the surgeon during surgical operations visual and data information useful to navigate, track and/or position implements, instrumentation, trial components, prosthetic components and other items and virtual constructs relative to the human body in order to improve performance of a repaired, replaced or reconstructed joint, and to do so in a manner that allows gross placement of cutting instruments followed by fine adjustment of cutting instruments through computer assisted navigation technology.

The embodiments of the present invention are better understood in reference to the following schematic drawings that are provided herein for illustrative purposes and are in no way limiting. The embodiments of the present invention may differ from the provided schematic illustrations.

### BRIEF DESCRIPTION OF THE DRAWINGS

FIG. 1 is an isometric representation of an embodiment of an input device for computer-assisted surgery.

FIG. 2 is an isometric view of the input device of FIG. 1 showing one of the fiducials displaced relative to another fiducial and to the body of the device.

FIG. 3 is a schematic representation of a second embodiment of a data input device.

FIG. 4 is a schematic representation of an operation of a data input devices during computer assisted surgery.

### DETAILED DESCRIPTION

The foregoing discloses preferred embodiments of the present invention, and numerous modifications or alterations may be made without departing from the spirit and the scope of the invention.

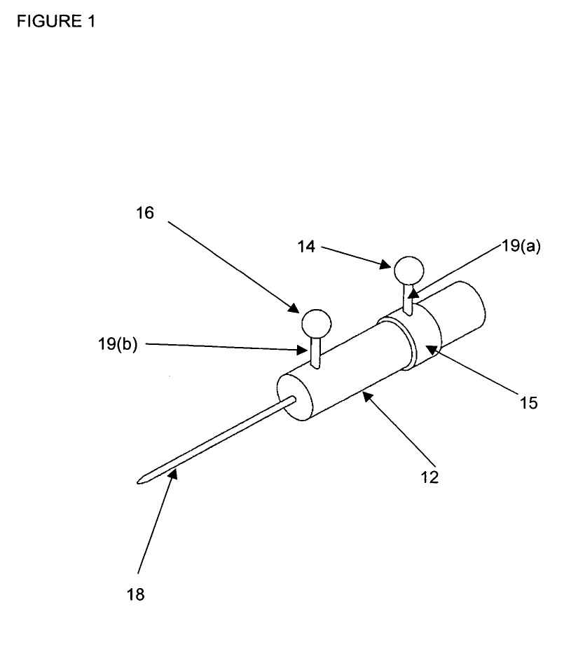

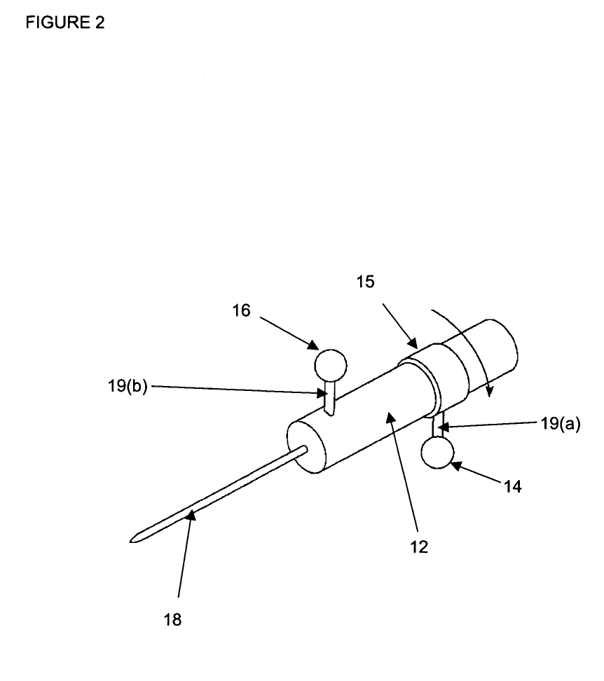

In a first embodiment, the device for data input is a probe (10) comprising a body (12) one or more markers or fiducials operatively associated with the probe and movable relative to the body of the probe (14). In the disclosed embodiment, the movable fiducial (14) is mounted to a rotatable collar (15) mounted to the body (12) of the probe (10). One or more other markers or fiducials (16) are operatively associated with the probe and stationary relative to the body of the probe (12). In one embodiment shown in FIGS. 1-2, the probe comprises two markers or fiducials (14, 16), wherein a first fiducial (14) is movable relative to the body (12) of the probe, and a second fiducial (16) is stationary relative to the body of the probe. The number of fiducials is not limited to that shown in FIGS. 1-2.

In a preferred embodiment, the body (12) of the probe (10) is elongated and comprises a pointed elongated tip (18). The movement of the one or more movable markers or fiducials (14) relative to the one or more stationary markers or fiducials (16) triggers the data input, as will be described in more detail below. In one embodiment, the probe further comprises one or more shafts (19(a) and 19(b)) associating the one or more movable fiducials (14), or the one or more stationary markers or fiducials (16), or both, to the body of the probe (12).

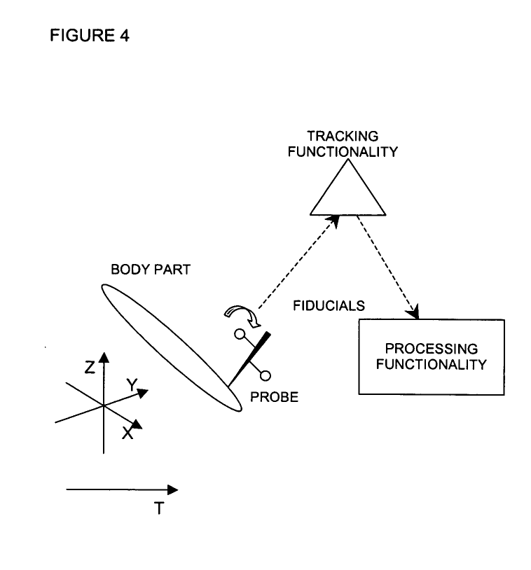

As illustrated in FIG. 4, a tracking or sensing functionality, such as a position and/or orientation sensor, tracks the position and/or orientation of the one or more of markers or fiducials by registering a signal from the fiducials. The tracking or sensing functionality, in turn, communicates to the processing functionality the signal from the probe. The processing functionality registers the signal from the probe and interprets it as data on position and/or orientation of the probe.

During computer-assisted surgery, a user locates the improved probe (10) on a patient's body structure. More specifically, the user manipulates the probe (10) to locate the point of the probe's tip (18) at a point on the body structure. It is to be understood that the term “point” is used to refer to a physical location and is not limited to a single geometrical point. The user then manipulates the probe to induce movement of the one or more movable markers or fiducials relative to the one or more stationary markers or fiducials. The movement of the one or more markers or fiducials relative to the to the one or more stationary markers or fiducials is registered by the processing functionality via the tracking or sensing functionality, which indicates to the processing functionality that the user is seeking to give input on the special and/or temporal location of the probe, more specifically, its tip, and/or its orientation.

In one embodiment, the fiducials operably associated with the probe are reflectors. The reflectors may be generally spherical or of other shapes. The reflectors may be of a variable shape. The reflectors may be manufactured of a reflective material, such as a reflective metal, non-metal, plastic, or ceramic material. Alternatively, the reflectors may comprise a reflective coating. In the embodiments where the shaft connects the one or more movable fiducials, the one or more stationary fiducials, or both to the body of the probe, the shafts can be manufactured of a reflective material or comprise a reflective coating.

In a preferred embodiment, the tracking or sensing functionality registers an infrared signal reflected by the reflective fiducials. Accordingly, the reflectors are adapted, although not limited, to reflect electromagnetic energy within the infrared spectrum. The tracking or sensing functionality can comprise one or more infrared cameras. Nevertheless, the position/orientation tracking sensors and fiducials need not be confined to the infrared spectrum. Any electromagnetic, electrostatic, light, sound, radiofrequency or other desired technique may be used if appropriate.

In the alternative, rather than the fiducials (14, 16) being passive, the fiducials can be active, that is, they can radiate infrared, electromagnetic, light, sound, radiofrequency, or other energy which can be sensed by the tracking or sensing functionality.

As noted above, the probe (10) comprises one or more fiducials (14) operably associated with the probe and movable relative to the body of the probe (12). In one example the one or more movable fiducials (14) are adapted to rotate around the body of the probe (12) as illustrated in FIG. 2. In one embodiment, the one or more movable fiducials (14) are associated with the probe's body by a shaft or a handle (19(a)), wherein the shaft or handle is adapted for rotation relative to the probe's body. Rotation of the shaft or the handle (19(a)) associated with the one or more movable fiducials (14) changes the position of the one or more movable fiducials (14) relative to the one or more stationary fiducials (16).

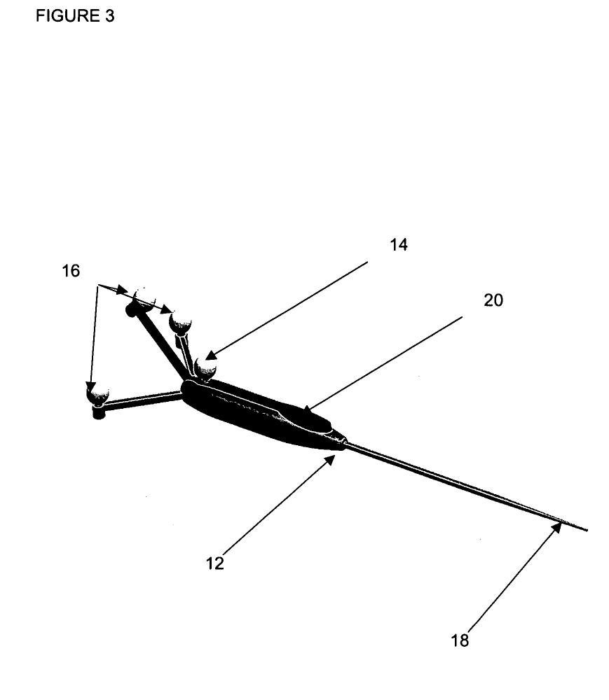

In another example, the one or more movable fiducials (14) elevate or lift relative to the body of the probe, thereby changing its position relative to the one or more stationary fiducials (16). In an embodiment illustrated in FIG. 3, a probe comprises a trigger mechanism (20). Actuating the trigger (20) moves the one or more movable fiducials (12) relative to the body of the probe (10).

It will be appreciated that the trigger mechanism can be adapted to move the movable fiducial in any of the rotational, radial, or axial directions upon actuation of the trigger. Similarly, the movable fiducial can be adapted for movement in response to manual manipulation in any of the rotational, radial, or axial directions.

In still another embodiment, the one or more movable fiducials is adapted to be axially displaced with respect to the handle. Thus the movable fiducial can move either rotationally, radially, or axially and be sensed by the tracking or sensing functionality, which in turn is registered by the processing functionality as a signal to capture data input.

The movement of the one or more movable fiducials relative to the one or more stationary fiducials indicates to the processing functionality that the user seeks to give input on the probe's position and/or orientation. In one embodiment, the user manipulates the probe to move the one or more movable fiducial to indicate a position and/or orientation. Upon input of the position and/or orientation data, the movable fiducials is allowed to return or is returned to its initial position. In another embodiment, when the user, for example, seeks to input a surface or the cloud of points, the user manipulates the probe to move the movable fiducial to indicate to the computer that the user is seeking input, and then allows the fiducial to remain in the position signaling input. The processing functionality then continuously registers the data on the position and/orientation of the probe until the movable fiducial is allowed to return or is returned to its initial position at the body of the probe.

The improved probe can be wireless and not require electrical power. The elimination of wires and a demand for a power supply, such as batteries, advantageously simplifies operation of the probe, including its preparation for the surgery, such as the requisite sterilization. The elimination and/or reduction of the electricity, radio waves, and other forms of energy capable of causing signal interference advantageously reduces interference with CAS or other sensitive equipment, such as imaging equipment, pacemakers and the like.

The improved probe is particularly well adapted for, although not limited to, minimally invasive surgery. In a preferred embodiment, the probe advantageously incorporates a pointed elongated tip allowing for discrete data point selection. The pointed elongated tip is also easy to use in minimally invasive surgical incisions. For data input, the tip remains stationary within the surgical incision, thereby avoiding unnecessary tissue trauma.

The improved probe may further include structures to move the one or more movable fiducials into more than one discrete position relative to the body of the probe, each position signaling to the processing functionality a category of input, such as, but not limiting to, whether to input a point or a surface. It is to be understood that either a point or a surface input may include any number of discrete data points on the probes position and/or orientation at temporal intervals. Differentiating between the categories of input may be accomplished by a variety of other means, such as incorporating into the probe several movable fiducials and changing the orientation of the markers separately or in any combination, assigning various combinations as indicative of an input category.

Instrumentation, systems, and processes according to a preferred embodiment of the present invention use computer capacity, including standalone and/or networked, to store data regarding spatial aspects of surgically related items and virtual constructs or references including body parts, implements, instrumentation, trial components, prosthetic components and rotational axes of body parts. Any or all of these may be physically or virtually connected to or incorporate any desired form of mark, structure, component, or other fiducial or reference device or technique which allows position and/or orientation of the associated item to be sensed and tracked, preferably in three dimensions of translation and three degrees of rotation as well as in time if desired. The fiducials may be tracked by one or more, preferably two, sometimes more infrared sensors whose output may be processed in concert to geometrically calculate position and orientation of the item to which the fiducial is attached.

During surgery, instrumentation, systems, and processes according to a preferred embodiment of the present invention employ a processing functionality, such as a computer, to register data on position and/or orientation of the probe to acquire information on the position and/or orientation of the patient's anatomical structures, such as certain anatomical landmarks, for example, a center of a femoral head. The information is used, among other things, to calculate and store reference axes of body components such as in a knee or a hip arthroplasty, for example, the axes of the femur and tibia, based on the data on the position and/or orientation of the improved probe. From these axes such systems track the position of the instrumentation and osteotomy guides so that bone resections position the prosthetic joint components optimally, usually aligned with a mechanical axis. Furthermore, the systems provide feedback on the balancing of the joint ligaments in a range of motion and under a variety of stresses and can suggest or at least provide more accurate information than in the past about the ligaments that the surgeon should release in order to obtain correct balancing, alignment and stability of the joint, improving patient's recovery. Instrumentation, systems and processes according to the present invention allow the attachment of a variable adjustor module so that a surgeon can grossly place a cutting block based on visual landmarks or navigation and then finely adjust the cutting block based on navigation and feedback from the system.

Instrumentation, systems, and processes according to the present invention can also suggest modifications to implant size, positioning, and other techniques to achieve optimal kinematics. Instrumentation, systems, and processes according to the present invention can also include databases of information regarding tasks such as ligament balancing, in order to provide suggestions to the surgeon based on performance of test results as automatically calculated by such instrumentation, systems, and processes.

The instrumentation, systems, and processes according to the present invention can be used in connection with computing functionality 18 which is networked or otherwise in communication with computing functionality in other locations, whether by PSTN, information exchange infrastructures such as packet switched networks including the Internet, or as otherwise desire. Such remote imaging may occur on computers, wireless devices, videoconferencing devices or in any other mode or on any other platform which is now or may in the future be capable of rending images or parts of them produced in accordance with the present invention. Parallel communication links such as switched or unswitched telephone call connections may also accompany or form part of such telemedical techniques. Distant databases such as online catalogs of implant suppliers or prosthetics buyers or distributors may form part of or be networked with functionality 18 to give the surgeon in real time access to additional options for implants which could be procured and used during the surgical operation.

The improved data input devices described herein are manufactured according to methods and principles known to those of skill in the art. The improved data input devices may incorporate synthetic materials, including, but not limited to, metals, ceramics, or plastics or combinations of them, various coatings, chemical elements and compounds. It is to be understood that the principles and structures of the data input devices illustrated herein are not limited to the surgical systems, devices, and application described herein, but can be applied to a variety of systems and devices, particularly medical systems and devices.

The particular embodiments of the invention have been described for clarity, but are not limiting of the present invention. Those of skill in the art can readily determine that additional embodiments and features of the invention are within the scope of the appended claims and equivalents thereto. All publications cited herein are incorporated by reference in their entirety.

1. A probe for data input for a computer-assisted surgical system, comprising:
a body having an axis;
one or more movable fiducials operably associated with the probe in movable relation thereto; and
one or more stationary fiducials operably associated with the probe in fixed relation thereto;
whereby the movement of the one or more movable fiducials relative to the one or more of the stationary fiducials triggers data input on a position or an orientation of the probe, or both.

2. The probe of claim 1, wherein the probe comprises a pointed elongated tip.

3. The probe of claim 2, wherein the pointed elongated tip is adapted for insertion through a surgical incision during computer assisted surgery.

4. The probe of claim 3, wherein the pointed elongated tip is adapted for insertion through a minimally invasive surgical incision.

5. The probe of claim 1, wherein the fiducials are passive fiducials.

6. The probe of claim 5, wherein the fiducials are reflectors.

7. The probe of claim 6 wherein the reflectors are infrared reflectors.

8. The probe of claim 1, wherein the fiducials are active fiducials.

9. The probe of claim 1, wherein the probe further comprises a shaft associating the one or more movable fiducials with the body of the probe.

10. The probe of claim 9, wherein the shaft is reflective.

11. The probe of claim 1, wherein the movable fiducial is axially movable with respect to the axis of the body.

12. The probe of claim 1, wherein the movable fiducial is rotationally movable with respect to the axis of the body.

13. The probe of claim 1, wherein the movable fiducial is radially movable with respect to the axis of the body.

14. The probe of claim 1, further comprising a mechanism for rotating the one or more movable fiducials relative to the body of the probe.

15. The probe of claim 1, further comprising a mechanism for displacing the one or more movable fiducials radially relative to the body of the probe.

16. The probe of claim 1, further comprising a mechanism for axially displacing the one or more movable fiducials relative to the body of the probe.

17. The probe of claim 1, further comprising an actuating mechanism, wherein triggering the actuating mechanism moves the one or more movable fiducials relative to the one or more stationary fiducials.

18. A system for computer-assisted surgery, comprising:
a probe for data input for a computer-assisted surgical system, comprising: a body; one or more movable fiducials operably associated with the probe; and one or more stationary fiducials operably associated with the probe; wherein the movement of the one or more movable fiducials relative to the one or more of the stationary fiducials triggers data input on a position or an orientation of the probe, or both;
a tracking functionality adapted for tracking the one or more movable markers or fiducials; and
a processing functionality adapted to receive from the tracking functionality signals on the motion of the one or more detector and interpreting the signals to input information on the location or the orientation, or both, of the probe.

19. The system of claim 18, wherein the body of the probe comprises a pointed elongated tip.

20. The system of claim 19, wherein the pointed elongated tip is adapted for insertion through a surgical incision during computer assisted surgery.

21. The system of claim 18, wherein the pointed elongated tip is adapted for insertion through a minimally invasive surgical incision.

22. The system of claim 18, wherein the fiducials are passive fiducials.

23. The system of claim 22, wherein the fiducials are reflectors.

24. The system of claim 23 wherein the reflectors are infrared reflectors.

25. The system of claim 18, wherein the fiducials are active fiducials.

26. The system of claim 18, wherein the probe further comprises a shaft associating the one or more movable fiducials with the body of the probe.

27. The system of claim 26, wherein the shaft is reflective.

28. The system of claim 18, further comprising a mechanism for rotating the one or more movable fiducials relative to the one or more stationary fiducials.

29. The system of claim 18, further comprising a mechanism for elevating the one or more movable fiducials relative to the one or more stationary fiducials.

30. The system of claim 18, further comprising a trigger, wherein actuating the trigger moves the one or more movable fiducials relative to the one or more stationary fiducials.

31. The system of claim 18, wherein the tracking functionality is an infrared detector.

32. A method of data input during computer assisted-surgery, comprising the steps of:
providing a probe for data input for a computer-assisted surgical system, comprising: a body; one or more movable fiducials operably associated with the probe; and one or more stationary fiducials operably associated with the probe; wherein the movement of the one or more movable fiducials relative to the one or more of the stationary fiducials triggers data input on a position or an orientation of the probe, or both;
providing a tracking functionality adapted to track the probe;
providing a processing functionality adapted to receive a signal from a tracking functionality and input data on a position or orientation of the probe, or both;
locating the probe on a body structure of a patient; and
manipulating the probe to induce movement of the one or more movable fiducials relative to the one or more stationary fiducials to indicate to the processing functionality that to input the location or orientation, or both, of the probe.

33. The method of claim 32, wherein the patient is a joint replacement surgery patient.

34. The method of claim 32, wherein the patient is a neurological surgery patient.

35. The method of claim 32, wherein the patient is an ear, nose and throat surgery patient.

36. The method of claim 32, further comprising in step of locating the probe, providing a minimally invasive surgical incision at a patient and inserting the probe through the minimally invasive surgical incision.

37. The method of claim 32, wherein the body structure is an anatomical landmark.

38. A probe for data input for a computer-assisted surgical system, comprising:
a body comprising a pointed elongated tip adapted for minimally invasive surgery;
one or more movable fiducials operably associated with the probe; and
one or more stationary fiducials operably associated with the probe;
wherein the movement of the one or more movable fiducials relative to the one or more of the stationary fiducials triggers data input on a position or an orientation of the probe, or both.
